(19) United States
(12) Patent Application Publication
(10) Pub. No.: US 2020/0164164 A1
(43) Pub. Date: May 28, 2020

(54) FLUID PRODUCT DISTRIBUTION DEVICE

(71) Applicant: APTAR FRANCE SAS, LE NEUBOURG (FR)

(72) Inventors: Michael HELMLINGER, Radolfzell (DE); Joachim KORNER, Uhldingen-Muhlhofen (FR)

(73) Assignee: APTAR FRANCE SAS, LE NEUBOURG (FR)

(21) Appl. No.: 16/630,060

(22) PCT Filed: Jul. 10, 2018
(86) PCT No.: PCT/FR2018/051733

§ 371 Date: Jan. 10, 2020

(30) Foreign Application Priority Data
Jul. 11, 2017 (FR) 1756579

(51) Int. Cl. A61M 15/00

(52) U.S. Cl. CPC A61M 15/0051; A61M 15/0021; A61M 15/004; A61M 2205/8206; A61M 15/0091; A61M 2202/064; A61M 15/008

(57) ABSTRACT

A fluid dispenser device with a main body (10); a reservoir (21) containing a dose of powder; a reservoir opening mechanism (80); a dispenser orifice (5); a movable support mechanism (50) to displace a reservoir (21) against the opening mechanism on actuation and displaceable between a non-dispensing position and a dispensing position, urged towards the dispensing position, and held in the non-dispensing position by a blocking mechanism (100). An inhalation trigger system (60) is provided that has a deformable air chamber (61) and a trigger element (600) that co-operates firstly with the air chamber (61) and secondly with the blocking mechanism. The fluid dispenser device further includes an electronic dose counter or indicator device (300) including a sensor (306) to detect displacement or deformation of a portion of the fluid dispenser device, generated while the user is inhaling or after the user has inhaled.

The present invention relates to a fluid dispenser device, and more particularly to a dry-powder inhaler.

Inhalers are well known in the prior art. Various types exist.

A first type of inhaler contains a reservoir receiving many doses of powder, the inhaler being provided with metering means making it possible, on each actuation, to remove one dose of said powder from the reservoir, so as to bring said dose into an expulsion duct in order to be dispensed to the user.

The prior art also describes inhalers including individual reservoirs, such as capsules, that are loaded into the inhaler just before said reservoir is used. The advantage of such devices is that it is not necessary to store all of the doses inside the appliance, such that said appliance can be compact. However, the inhaler is more difficult to use, since the user is obliged to load a capsule into the inhaler before each use.

Another type of inhaler consists in placing the doses of powder in individual predosed reservoirs, then in opening one of the reservoirs each time the inhaler is actuated. That implementation seals the powder more effectively since each dose is opened only when it is about to be expelled. In order to make such individual reservoirs, various techniques have already been proposed, such as an elongate blister strip or blisters arranged on a rotary circular disk.

All existing types of inhalers, including those described above, present both advantages and drawbacks associated with their structures and with their types of operation.

Thus, with certain inhalers, there is the problem of metering accuracy and reproducibility on each actuation.

In addition, the effectiveness of the dispensing, i.e. the fraction of the dose that effectively penetrates into the user's lungs in order to have a beneficial therapeutic effect, is also a problem that exists with a certain number of inhalers.

A solution for solving that specific problem has been to synchronize the expulsion of the dose with the inhalation of the patient. Once again, that can create drawbacks, in particular in that type of device, the dose is generally initially loaded into an expulsion duct before inhalation, then expulsion is synchronized with inhalation. That means that if the user drops, shakes, or manipulates the inhaler in an undesirable or inappropriate manner between the moment when the user loads the dose (either from a multidose reservoir or from an individual reservoir) and the moment when the user inhales, then the user risks losing all or part of the dose, with said dose possibly being spread about inside the appliance. In that event, there can exist a high risk of overdosing the next time the device is used. The user who realizes that the dose is not complete will load a new dose into the appliance, and while the new dose is being inhaled, a fraction of the preceding dose that was lost in the appliance could thus be expelled at the same time as the new dose, thereby causing an overdose. In the treatments envisaged, such overdosing can be very harmful, and the authorities in all countries are issuing ever-stricter requirements to limit the risk of overdosing as much as possible.

Another problem that may occur relates to assembling certain parts, in particular movable parts, that need to withstand large stresses in operation, and for which assembly needs to be particularly reliable so as to avoid any risk of malfunctioning. With the small size of certain parts and possible deformations resulting from molding and/or from conditioning plastic components, it can be complicated to guarantee such reliable assembly. Leaktight assemblies are desirable, in particular in inhalers using an inhalation trigger system, in particular so as to avoid any head loss during inhalation, and also so as to ensure that the trigger threshold is repeatable. This improves comfort in use for patients, and makes it possible to avoid disturbing the habits of patients in the event of variations from one inhaler to another.

In addition, it is known to use a dose counter or indicator for informing the user about the number of doses that have been dispensed or that remain to be dispensed. Such counters or indicators are generally mechanical. A conventional drawback with such mechanical counters is that they are bulky, correspondingly increasing the size of the inhaler itself. Furthermore, the display is very small and often difficult to read, in particular for the elderly. In particular, this is true for counters for counting a high number of doses, e.g. 30 or 60 doses. Another problem associated with counting doses relates to the counters or indicators in which actuation is not associated directly with dispensing a dose of powder, but with an actuation stage such as loading the dose to be dispensed or the like. If for some reason the user does not end up inhaling the loaded dose, said dose will still be counted.

Documents U.S. Pat. No. 6,179,164, US 2005 174216, FR 2 936 424, FR 2 936 425, WO 02/085281, WO 2008/012458, WO 2009/077697, and WO 2011/154659 describe prior-art devices.

An object of the present invention is to provide a fluid dispenser device, in particular a dry-powder inhaler, that does not have the above-mentioned drawbacks.

In particular, an object of the present invention is to provide such a device that can be assembled and used reliably, guaranteeing metering accuracy and reproducibility on each actuation, providing an optimum yield with regard to the effectiveness of the treatment, by making it possible to dispense a substantial fraction of the dose to the zones to be treated, in particular to the lungs, while avoiding, in safe and effective manner, any risk of overdosing, and that is as compact as possible, while guaranteeing sealing and absolute integrity of all of the doses up to their expulsion.

Another object of the present invention is to provide such a device that guarantees reliable and accurate counting on each actuation, preventing any risk of under-counting and/or of over-counting.

In addition, an object of the present invention is to provide such a device that is simple and inexpensive to manufacture and to assemble.

The present invention thus provides a fluid dispenser device including a main body, said device comprising:
at least one individual reservoir containing a single dose of fluid, such as powder;
opening means for opening an individual reservoir each time the device is actuated;
a mouthpiece defining a dispenser orifice;
movable support means that are adapted to displace an individual reservoir against said opening means on each actuation, said movable support means being displaceable between a non-dispensing position and a dispensing position, said movable support means being urged towards their dispensing position by resilient means, such as a spring or a spring blade, and being held in their non-dispensing position by blocking means; and
an inhalation trigger system that comprises a deformable air chamber that co-operates with said dispenser orifice, and a trigger element that co-operates firstly with said air chamber and secondly with said blocking means, so that during inhalation through said dispenser orifice, said air chamber is deformed and said trigger element releases said blocking means, so that during inhalation, a reservoir is displaced against said opening means and is opened by said opening means;

said fluid dispenser device further comprising an electronic dose counter or indicator device including a sensor that is adapted to detect the displacement or the deformation of a portion of said fluid dispenser device, generated while the user is inhaling or after the user has inhaled.

Advantageously, said sensor is actuated by said movable support means while they are being displaced between said non-dispensing position and said dispensing position.

In a variant, said sensor is actuated by said movable support means while they are being displaced between said dispensing position and said non-dispensing position.

In another variant, said sensor is actuated by said blocking means when they are released from their blocking position.

In still another variant, said sensor is actuated by the displacement and/or the deformation of said trigger element.

In still another variant, said sensor is actuated by the displacement and/or the deformation of said deformable air chamber.

Advantageously, the device includes an elongate strip of individual reservoirs co-operating with first displacement means that are adapted to cause said strip to advance after each actuation, said first displacement means comprising a guide wheel that is rotatably mounted on said movable support means.

Advantageously, said sensor is actuated by said guide wheel turning.

Advantageously, said electronic dose counter or indicator device comprises a screen, in particular of the liquid crystal display (LCD) type, a power supply, such as an optionally rechargeable battery, and a printed circuit.

Advantageously, said sensor, when it is actuated, transmits a signal to said printed circuit, which signal causes the display on said screen to be changed.

Advantageously, the device includes a cocking member that is displaced towards a cocked (i.e. spring-loaded) position while opening the fluid dispenser device, said electronic dose counter or indicator device including an actuator that is provided with a connector, and said cocking member including an extension that is adapted to co-operate, in the cocked position of said cocking member, with said connector so as to activate the electronic dose counter or indicator device.

Advantageously, said electronic dose counter or indicator device includes a memory that is adapted to store the information generated by said sensor.

Advantageously, said electronic dose counter or indicator device includes wireless data transmission means, in particular of the Bluetooth® type, in particular of the Bluetooth® low energy (BLE) type.

Advantageously, said electronic dose counter or indicator device includes other sensors associated with said sensor, in particular sensors of the accelerometer type, for detecting the orientation and/or the movements of the user while said fluid dispenser device is being actuated.

Advantageously, said opening means include a perforator element that is stationary relative to said main body and that is adapted to cut a closure wall of the reservoir in such a manner that the cut portion(s) does/do not obstruct the opening(s) that is/are formed.

Advantageously, the device includes at least one cover element that is mounted to pivot on said main body between a closed position and an open position.

These characteristics and advantages and others of the present invention appear more clearly from the following detailed description, given by way of non-limiting examples, and with reference to the accompanying drawings, and in which:

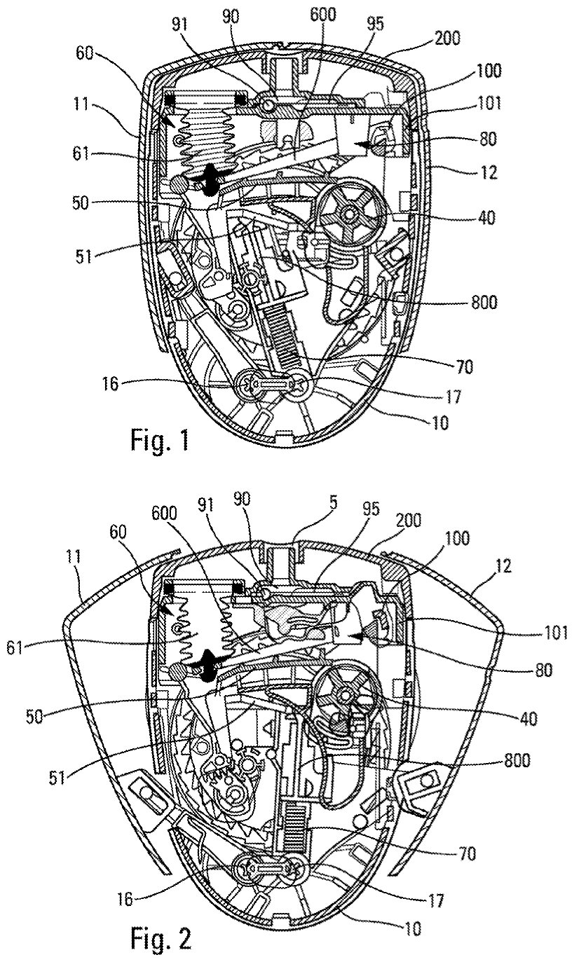

FIG. 1 is a diagrammatic section view of a dispenser device in an advantageous embodiment of the invention, in its rest position, before opening and cocking the device;

FIG. 2 is a view similar to the view in FIG. 1, in its cocked position, before inhalation;

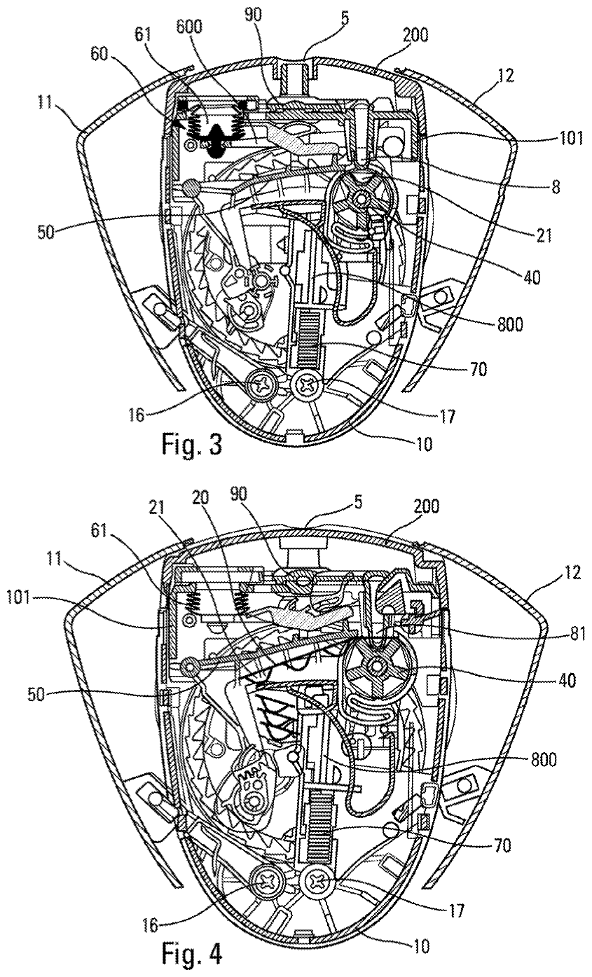

FIG. 3 is a view similar to the view in FIG. 2, during inhalation;

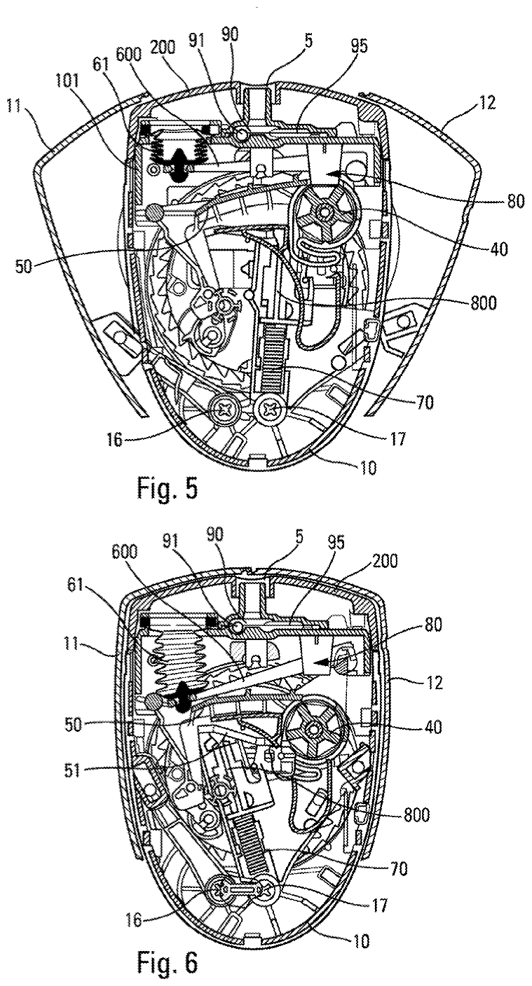

FIGS. 4 and 5 are two views similar to the view in FIG. 3, at the end of inhalation;

FIG. 6 is a view similar to the view in FIG. 5, in the final position, after the device has been closed;

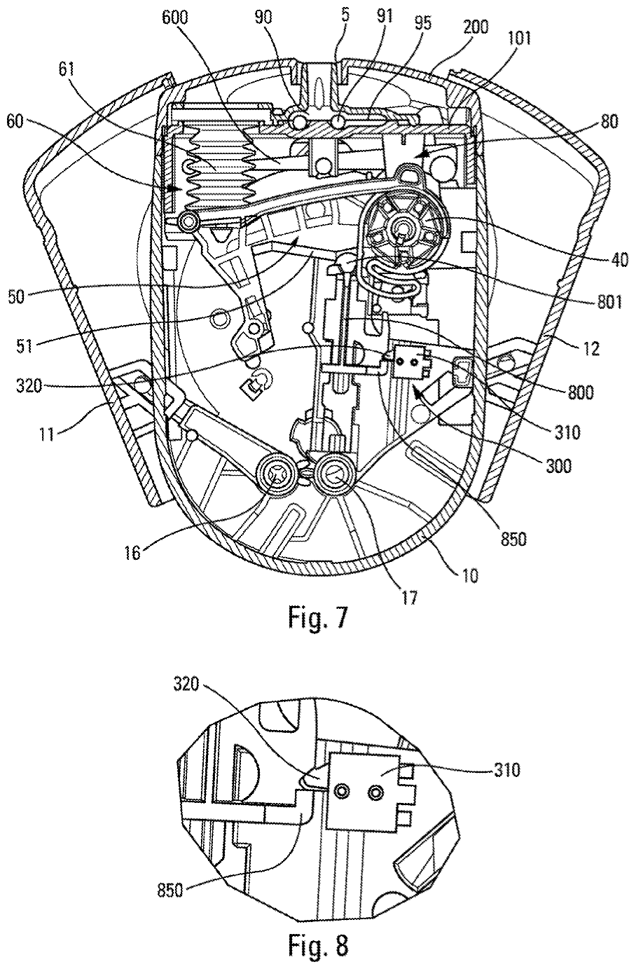

FIG. 7 is a diagrammatic and partially cut-away perspective view, in the cocked position, before inhalation;

FIG. 8 is view of a detail in FIG. 7; et

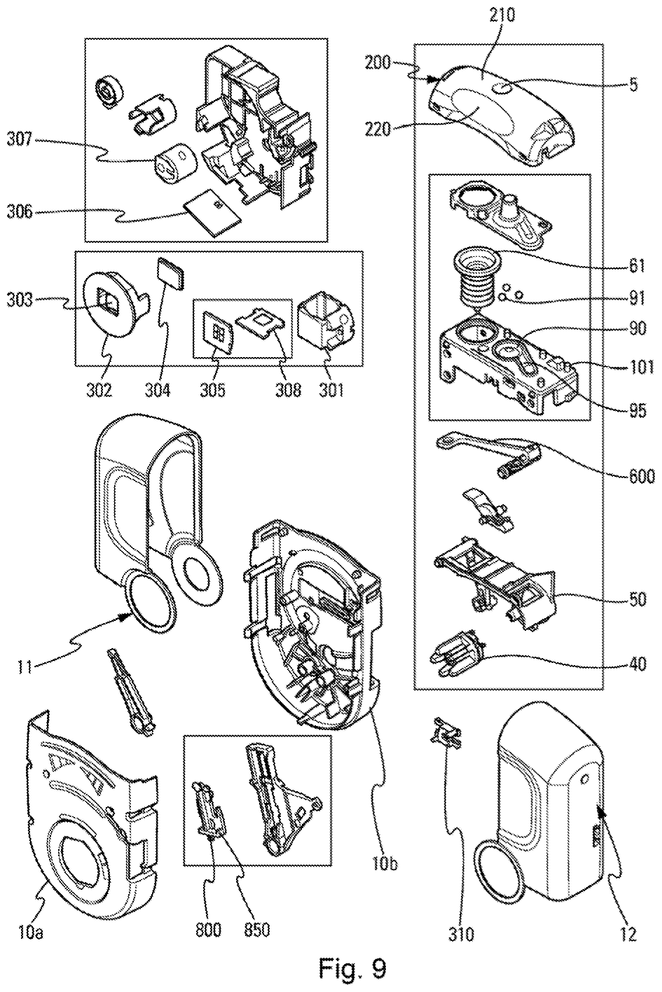

FIG. 9 is an exploded diagrammatic and fragmentary perspective view of the component elements of the dispenser device in FIGS. 1 to 8.

The figures show an advantageous embodiment of a dry-powder inhaler. In FIG. 9, only the elements mentioned in the description below are given numerical references.

The inhaler shown in the figures includes a main body 10 on which there can be slidably mounted two cover-forming portions 11, 12 that are adapted to be opened so as to open and cock (i.e. spring-load) the device. The main body 10 comprises two body portions 10a and 10b that are assembled together, as shown in FIG. 9, and it can be approximately rounded in shape, as shown in the figures, but it could be of any other appropriate shape. An upper body 101 is assembled to the main body 10, and a mouthpiece 200 is assembled on said upper body 101. The mouthpiece 200 defines a dispenser orifice 5 through which the user inhales while the device is being actuated. The dispenser orifice 5 is typically arranged approximately in the center of the mouthpiece 200. The covers 11, 12 can open by pivoting about a common pivot axis, or about two parallel axes 16, 17, as shown in the figures, e.g. by meshing together. Any other opening means for opening the device can be envisaged. In a variant, the device could include only a single cover instead of two.

Inside the main body 10 there is provided a strip 20 of individual reservoirs 21 (shown only diagrammatically in FIG. 4, for the sake of clarity), also known as blisters, said strip being made in the form of an elongate strip on which the blisters are arranged one behind another, in manner known per se. The blister strip 20 is advantageously constituted by a base layer or wall that forms the cavities receiving the doses of powder, and by a closure layer or wall that covers each of said blisters in sealed manner. Before first use, the blister strip can be rolled-up inside the main body 10, preferably in a storage portion, and first strip displacement means 40, in particular rotary means, are provided for progressively unrolling the blister strip and for causing it to advance.

Second displacement means 50, preferably formed by movable support means comprising a pivot member 50 that pivots on the main body 10, are provided for bringing a respective blister 21 into a dispensing position each time the device is actuated. The second displacement means are advantageously mounted to pivot between a non-dispensing position and a dispensing position in which a blister co-operates with opening means 80.

The inhaler includes blister opening means 80 comprising a perforator and/or cutter element 81 for perforating and/or cutting the closure layer of the blisters. Preferably, the perforator element 81 is stationary relative to the main body 10 and the upper body 101. A respective blister 21 is displaced on each actuation against said perforator element 81 by the second displacement means 50. The blister 21 is thus perforated by said perforator element 81 that penetrates into said blister so as to expel the powder by means of the user inhaling, as shown in FIGS. 4 and 5. Advantageously, the opening means 80 are adapted to cut a closure wall of the reservoir or blister 21 in such a manner that the cut portion(s) does/do not obstruct the opening(s) that is/are formed. Documents WO 2006/079750 and WO 2009/007640 describe such blister opening means.

The first displacement means 40 are adapted to cause the blister strip to advance after each inhalation of the user. The second displacement means 50 are adapted to displace the blister that is to be emptied against said opening means 80 during actuation, before each inhalation. The second displacement means 40 can be urged by a resilient element 70, such as a spring or any other equivalent resilient element, said resilient element being suitable for being prestressed while the device is being opened.

Preferably, the first displacement means 40 are formed by an indexer wheel that receives and guides the blister strip. The description below is thus made with reference to such an indexer wheel 40. Turning the indexer wheel 40 causes the blister strip to advance. Before each inhalation, a full blister is always in a position facing the opening means 80. The second displacement means 50 can include a pivot member that is mounted to pivot about a pivot axis, said indexer wheel 40 advantageously being rotatably mounted on said pivot member.

An actuation cycle of the device can be as follows. While the device is being opened, the two cover-forming lateral portions 11, 12 are moved away from each other by pivoting about the body 10 so as to open the device and thus cock the device. In this position, shown in FIG. 2, the indexer wheel 40 cannot be displaced towards the perforator element 80, since the second displacement means 50 are held by appropriate blocking means 100 (shown only very diagrammatically for the sake of clarity). Documents WO 2009/077700 and WO 2009/136098 describe such opening means. While the user is inhaling through the mouthpiece, the blocking means 100 are unblocked, thereby causing said indexer wheel 40 to be displaced towards the perforator element 81, and thereby causing a blister 21 to be opened.

As explained above, it is desirable for the opening means 80 to be actuated by the user inhaling. In order to trigger the opening means 80 by inhalation, an inhalation trigger system 60 is provided that advantageously comprises an air chamber 61 that is deformable under the effect of inhalation, the air chamber being adapted to release the blocking means 100. The air chamber 61 may advantageously be made in the form of a bellows. Inhalation by the user causes said deformable air-chamber 61 to deform, thereby releasing said blocking means 100 and enabling the second displacement means 50 to be displaced, and therefore enabling a respective blister 21 to be displaced towards its opening position. The blister 21 is therefore opened only on inhalation, such that it is emptied simultaneously. Thus, there is no risk of any of the dose being lost between opening the blister and emptying it.

The inhaler may further include a dispersion chamber 90 for receiving the dose of powder after a respective blister 21 has been opened. The dispersion chamber 90 is advantageously provided with at least one and preferably more beads 91 that are displaced inside said dispersion chamber 90 during inhalation, in particular so as to improve dispensing of the air and powder mixture after a blister has been opened, in order to increase the effectiveness of the device.

It can be advantageous for the opening means 80, in particular for the perforator element 81, to be connected directly to said dispersion chamber 90, e.g. via a channel 95 leading to said chamber 90.

After inhalation, when the user closes the device, all of the components return to their initial rest positions. The device is thus ready for a new utilization cycle.

In an advantageous aspect of the inhaler, the blisters are formed on a flexible elongate strip that, initially, is mainly stored in the form of a roll in a storage housing inside the main body 10 of the device. Advantageously, the rolled-up blister strip is held by inner walls of said storage housing without its rear end (rear in the direction of advance of the blister strip) being fastened relative to said main body 10, thereby enabling the blister-strip roll to be assembled more easily inside the device. The blister strip is displaced by means of the indexer wheel 40 that advantageously presents at least one and preferably more recesses, each having a shape that corresponds to the shape of the blisters. Thus, when the indexer wheel 40 turns, it causes the blister strip to advance. Naturally, in a variant or in additional manner, it is possible to use other means for advancing the blister strip, e.g. providing a profile on the longitudinal lateral edges of the blister strip, said profile being adapted to co-operate with appropriate drive means. In addition, holes formed along the lateral edges of the blister strip could also be used to cause the blister strip to advance by means of sprocket wheels co-operating with said holes.

After opening one or more blisters, the blister-strip portion with the empty blisters must be suitable for being stored in easy and compact manner in the device, while avoiding any risk of blockage. Advantageously, the used blister strip is rolled-up automatically, once again forming a roll.

The movable cover element 12 is secured to a cocking member 800 that can slide in an appropriate housing. The cocking member 800 thus advantageously pivots relative to said body 10 together with the cover element 12. The cocking member 800 may be displaced against a spring 70, advantageously a coil spring, that is arranged in said housing. The cocking member 800 is thus connected at one end to said spring 70, and at the other end it co-operates with the second displacement means, in particular with a pivot member 50 that is mounted to pivot on the body 10, and on which the indexer wheel 40 is fastened in rotary manner.

When the movable cover element 12 is opened, the cocking member 800 is displaced in its housing, compressing the spring 70. The pivot member 50 of the second displacement means is itself prevented from being displaced by the above-mentioned blocking means 100 that are released only at the moment of inhalation. Thus, in the absence of any inhalation in the open position, closing the cover elements 11, 12 would merely cause the cocking member 800 to return to its rest position and the spring 70 to decompress.

Thus, by opening the inhaler, the user cocks the system. If the user does not inhale and closes the inhaler, said inhaler merely returns to its start position without displacing the blister strip or the blocking means. There is thus no risk of a blister (and thus an active dose of substance) being lost by accidental or incomplete actuation in which the user does not inhale between opening and closing. Opening the blister, emptying it, dispensing the powder into the lungs of the user, displacing the blister strip to bring a new full blister to face the opening means, and counting the dose are thus possible only if the user inhales.

The blocking means 100 that block the second displacement means and in particular the pivot member 50 that co-operates with the cocking member 800, are connected to the deformable air chamber 61 that is sensitive to the user inhaling, via a trigger element 600, so that while the user is inhaling, said deformable air chamber 61 deforms, causing the trigger element 600 to pivot and causing said blocking means 100 to be released. This enables said second displacement means 50 to be displaced towards their dispensing position under the effect of the force exerted by the compressed spring 70 on the cocking member 800 that pushes against the pivot member 50. Such displacement causes a full blister to be opened and a dose to be dispensed.

A cam surface 51 is formed on said movable support means 50, on which the cocking member 800 slides. The cocking member 800 is thus adapted to compress the spring 70 when the cover element 12 is opened, and to decompress said spring 70 when said cover element 12 is closed.

Advantageously, in its portion in contact with the cam surface 51, the cocking member 800 includes a rounded portion 801 for facilitating sliding of the cocking member 800 on said cam surface 51.

In this embodiment, the movable support means are made in the form of a member 50 that is pivotally mounted on the body 10 about a pivot axis. Since the above-mentioned cam surface 51 is formed on said pivot member 50, when the spring 70 is stressed while the movable cap element 12 is opening, said pivot member 50 is urged towards its dispensing position by said cocking member 800 and the spring 70 is compressed.

The cam surface 51 may include at least two portions of different slopes that are advantageously separated by a vertex. Starting once again from the closed position of the movable cover element, the first slope portion on which the cocking member 800 slides, enables the spring 70 to be compressed, as described above. When the spring is stressed, i.e. compressed, the cam surface 51 may provide a second different slope portion with which the cocking member 800 co-operates only when the device is in its open position. The cocking member 800 preferably exerts a force that is substantially perpendicular on the second cam surface portion. In this way, the cocked position is stable. In a variant, the second slope portion forms an abutment notch in which the cocking member 800 comes to be positioned in the open position.

After inhalation, i.e. in the dispensing position, the blocking means have been released, and the movable support means 50 have been displaced upwards by the compressed spring 70.

Advantageously, the two movable cover elements 11, 12 mesh together via appropriate gearing so as to guarantee symmetrical opening and closing of said two movable cover elements. They can mesh together in the proximity of their pivot axes 16, 17.

The mouthpiece 200 is preferably symmetrical about said dispenser orifice 5, as can be seen in FIG. 9. It includes a top wall 210 that extends longitudinally on both sides of the dispenser orifice 5. It should be observed that the top wall is slightly humped or convex in the longitudinal direction, thereby imparting a flattened rounded shape to the mouthpiece 200. On either side of the dispenser orifice 5, in the transverse or lateral direction, the mouthpiece includes two lateral bearing surfaces 220 that are respectively provided laterally on each side of said dispenser orifice 5. Each of the lateral bearing surfaces 220 is for receiving a lip of the user during inhalation. The lateral bearing surfaces 220 are slightly concave, in particular in the transverse direction, so as to form slight hollows that are adapted to position the lips of the user properly, when said user places them on the mouthpiece. As can be seen in FIG. 9, the lateral bearing surfaces 220 extend said top wall 210 laterally, and they are preferably spaced apart from each other by the top wall 210 and by the dispenser orifice 5. In this way, when the user places the mouth on the mouthpiece 200, the spacing between the two lateral bearing surfaces 220 obliges the user to open the mouth wide enough to avoid the teeth obstructing the dispenser orifice 5, even in part, during inhalation. In addition, the slightly concave shape of the lateral bearing surfaces 220 is ergonomic, ensuring good sealing with the mouth of the user on the mouthpiece 200 during inhalation. In particular, this avoids head loss during inhalation, and guarantees that as much as possible of the user-created inhalation flow is used to actuate the inhaler and to expel the powder contained in the inhaler. The spacing and the depth of the lateral bearing surfaces 220 also guarantee that the dispenser orifice is positioned inside the mouth, beyond the teeth, but without the dispenser orifice 5 being formed on a projecting portion of the mouthpiece 200.

The lateral bearing surfaces 220 advantageously extend longitudinally over a central portion of said mouthpiece 200, preferably over the major part thereof, as shown in particular in FIG. 9. Advantageously, the two lateral bearing surfaces 220 are completely symmetrical to each other, about the dispenser orifice 5.

Advantageously, the cover elements 11, 12 that are movable about the main body 10 of the inhaler and about the mouthpiece 200, include a top surface of convex shape that is substantially adapted to the shape of the mouthpiece, in particular at its top wall 210. In this way, while said cover elements are being displaced between the open and closed positions, no gap is created as a result of the presence of the mouthpiece 200, such that the fingers of the user do not risk being pinched while manipulating the cover elements. In general, the mouthpiece does not include any projecting portion that risks creating a gap that could receive the user's fingers while the device is being actuated.

In the invention, an electronic dose counter or indicator device 300 is provided. The device may include a screen that displays numbers or symbols that are visible through an appropriate window in the main body 10 of the device.

An object of the invention is to avoid counting doses that have not been dispensed, e.g. in the event of a manipulation error, or of an incomplete manipulation of the device. It is thus desirable that the counter or indicator is actuated only in the event of inhalation, since it is this inhalation that makes it possible for the blister to open and the dose contained therein to be dispensed. Advantageously, the counter is thus actuated during or after inhalation.

FIGS. 7 to 9 show an advantageous embodiment of the electronic dose counter or indicator device 300. In this embodiment, the electronic dose counter or indicator device 300 includes a body 301, and a cover 302 that is provided with a window 303 that is closed by a transparent wall 304. However, this embodiment is not limiting, and the counter 300 could merely be housed in an appropriate portion of the body 10 of the inhaler.

The electronic dose counter or indicator device 300 includes a screen 305, in particular of the liquid crystal display (LCD) type, a sensor 306, a power supply 307, such as an optionally rechargeable battery, and a printed circuit 308. It should be observed that the various elements could be made in ways that are different from that which is shown by way of example in the drawings.

Advantageously, provision is made for an actuator 310 that is provided with a connector 320. The cocking member 800 thus advantageously includes an extension 850 that is adapted to co-operate in the cocked position of the cocking member 800 with said connector so as to activate the electronic dose counter or indicator device 300. Thus, before the inhaler is opened and before the cocking member 800 is cocked, the electronic dose counter or indicator device 300 is preferably on standby so as to conserve the power supply 307. When the inhaler is opened and the cocking member 800 is cocked, the extension 850 of the cocking member 800 co-operates with the connector 320 of the actuator 310, which activates the electronic dose counter or indicator device 300, as can be seen in FIGS. 7 and 8.

At this stage, if the user closes the inhaler without inhaling, the electronic dose counter or indicator device 300 is not actuated, but is merely deactivated. No dose is counted.

However, if the user inhales, the electronic dose counter or indicator device 300 is actuated so as to count one dose and display it on the screen 305.

Actuation of the counter is thus correlated to an event, such as the displacement or the deformation of a part of the inhaler, that takes place during or after inhalation. Specifically, whenever the user inhales through the mouthpiece, inevitably a dose of powder is dispensed, and it must thus be counted by the counter.

Thus, the sensor 306 can detect the movement of the movable support means 50 which displace the reservoir that is to be emptied against the perforator element 81 after inhalation.

In a variant, the sensor 306 could detect the return of the movable support means 50, after the reservoir has been emptied by inhalation.

In another variant, the sensor 306 could detect the blocking means 100 being released and/or the trigger element 600 being displaced/deformed.

Still another variant would be to detect the displacement/deformation of the deformable air chamber 61.

Finally, when the first displacement means are actuated after inhalation, in particular while closing the inhaler, the sensor 306 could then also detect such displacement, in particular turning of the guide wheel 40.

The sensor 306, when it is actuated in this way, transmits a signal to said printed circuit, which signal causes the display on the screen 305 to be changed.

The signal may also be stored in an appropriate memory, together with timestamp and/or geolocation information. Advantageously, the device includes wireless data transmission means, in particular of the Bluetooth® type, in particular of the Bluetooth® low energy (BLE) type, for transmitting such information, e.g. to a smart phone or the like.

It should be observed that other sensors could be associated with the sensor 306, e.g. of the accelerometer type, for detecting the orientation and/or the movements of the user while using the inhaler, so as to complete the information relating to dose counting.

The present invention therefore makes it possible to provide a dry-powder inhaler that provides the following features:
a plurality of individual doses of powder stored in individual sealed blisters, e.g. 30 or 60 doses stored on a rolled-up strip;
the powder is released by perforation that is achieved by the user inhaling, the blister being perforated by means of an inhalation detector system that is coupled to a previously cocked release system;
appropriately-shaped drive means that are engaged with blisters so as to displace the blister strip after each inhalation, and bring a new full blister into a position in which it is to be opened by appropriate opening means;
means for avoiding doses being lost in the event of the inhaler being opened, but in the absence of any inhalation;
a reliable dose indicator adapted to count the doses only in the event of inhalation.

Other features are also provided by the device of the invention as described above.

It should be observed that the various features, even if they are shown as being provided simultaneously on the inhaler, could be implemented separately. In particular, the inhalation trigger mechanism could be used regardless of the type of reservoir opening means, regardless of the use of a dose indicator, regardless of the way in which the individual blisters are arranged relative to one another, etc. The cocking means and the inhalation trigger system could be made in some other way. The same applies for other component parts of the device.

Various modifications are also possible for the skilled person without departing from the scope of the present invention as defined in the accompanying claims. In particular, the various characteristics and functions of the device described with reference to the drawings can be combined together in any appropriate manner.

1.-16. (canceled)

17. A fluid dispenser device including a main body (10), said device comprising:
at least one individual reservoir (21) containing a single dose of fluid, such as powder;
opening means (80) for opening an individual reservoir each time the device is actuated;
a mouthpiece (200) defining a dispenser orifice (5);
movable support means (50) that are adapted to displace an individual reservoir (21) against said opening means (80) on each actuation, said movable support means (50) being displaceable between a non-dispensing position and a dispensing position, said movable support means (50) being urged towards their dispensing position by resilient means (70), such as a spring or a spring blade, and being held in their non-dispensing position by blocking means (100); and
an inhalation trigger system (60) that comprises a deformable air chamber (61) that co-operates with said dispenser orifice (5), and a trigger element (600) that co-operates firstly with said air chamber (61) and secondly with said blocking means (100), so that during inhalation through said dispenser orifice (5), said air chamber (61) is deformed and said trigger element (600) releases said blocking means (100), so that during inhalation, a reservoir is displaced against said opening means (80) and is opened by said opening means (80);
said fluid dispenser device further comprises an electronic dose counter or indicator device (300) including a sensor (306) that is adapted to detect the displacement of said movable support means (50) while the user is inhaling or after the user has inhaled.

18. A device according to claim 17, wherein said sensor (306) is actuated by said movable support means (50) while they are being displaced between said non-dispensing position and said dispensing position.

19. A device according to claim 17, wherein said sensor (306) is actuated by said movable support means (50) while they are being displaced between said dispensing position and said non-dispensing position.

20. A device according to claim 17, including an elongate strip (20) of individual reservoirs (21) co-operating with first displacement means (40) that are adapted to cause said strip to advance after each actuation, said first displacement means comprising a guide wheel (40) that is rotatably mounted on said movable support means (50).

21. A device according to claim 20, wherein said sensor (306) is actuated by said guide wheel (40) turning.

22. A device according to claim 17, wherein said electronic dose counter or indicator device (300) comprises a screen (305), in particular of the LCD type, a power supply (307), such as an optionally rechargeable battery, and a printed circuit (308).

23. A device according to claim 22, wherein said sensor (306), when it is actuated, transmits a signal to said printed circuit (308), which signal causes the display on said screen (305) to be changed.

24. A device according to claim 17, including a cocking member (800) that is displaced towards a cocked position while opening the fluid dispenser device, said electronic dose counter or indicator device (300) including an actuator (310) that is provided with a connector (320), and said cocking member (800) including an extension (850) that is adapted to co-operate, in the cocked position of said cocking member (800), with said connector (320) so as to activate the electronic dose counter or indicator device (300).

25. A device according to claim 17, wherein said electronic dose counter or indicator device (300) includes a memory that is adapted to store the information generated by said sensor (306).

26. A device according to claim 17, wherein said electronic dose counter or indicator device (300) includes wireless data transmission means, in particular of the Bluetooth® type, in particular of the Bluetooth® low energy (BLE) type.

27. A device according to claim 17, wherein said electronic dose counter or indicator device (300) includes other sensors associated with said sensor (306), in particular sensors of the accelerometer type, for detecting the orientation and/or the movements of the user while said fluid dispenser device is being actuated.

28. A device according to claim 17, wherein said opening means (80) include a perforator element (81) that is stationary relative to said main body (10) and that is adapted to cut a closure wall of the reservoir (21) in such a manner that the cut portion(s) does/do not obstruct the opening(s) that is/are formed.

29. A device according to claim 17, including at least one cover element (11, 12) that is mounted to pivot on said main body (10) between a closed position and an open position.
